(19) United States
(12) Patent Application Publication
(10) Pub. No.: US 2019/0307460 A1
(43) Pub. Date: Oct. 10, 2019

(54) INTRASACULAR OCCLUSION DEVICES METHODS PROCESSES AND SYSTEMS

(71) Applicant: Neurvana Medical, LLC, Tustin, CA (US)

(72) Inventors: David FERRERA, Coto de Daza, CA (US); Joshua BENJAMIN, Mission Viejo, CA (US)

(21) Appl. No.: 15/755,071

(22) PCT Filed: Aug. 24, 2016
(86) PCT No.: PCT/US2016/048486

§ 371 Date: Jun. 13, 2018

Related U.S. Application Data
(60) Provisional application No. 62/283,223, filed on Aug. 24, 2015.

(51) Int. Cl. A61B 17/12; A61B 17/221; A61L 31/02

(52) U.S. Cl. CPC A61B 17/12113; A61B 17/12172; A61B 2017/00526; A61B 17/12163; A61L 31/022; A61B 17/221

(57) ABSTRACT

Aneurysm embolization devices made from laser cut NITINOL types of metals are highly differentiated by their form, which governs function, in addressing acute states of aneurysm to achieve improved clinical outcomes, namely better results with fewer devices needed, alone or in complement with conventional coils and devices.

### BACKGROUND OF THE INVENTION

The pathological course of a blood vessel that is blocked is a gradual progression from reversible ischemia to irreversible infarction (cell death). A stroke is often referred to as a “brain attack” and occurs when a blood vessel in the brain becomes blocked or ruptures. An ischemic stroke occurs when a blood vessel in the brain becomes blocked. Occlusions may be partial or complete, and may be attributable to one or more of emboli, thrombi, calcified lesions, atheroma, macrophages, lipoproteins, any other accumulated vascular materials, or stenosis. Ischemic strokes account for about 80% of all strokes. Hemorrhagic strokes, which account for the remaining 20% of strokes, occur when a blood vessel in the brain ruptures. Stroke is the third leading cause of death in the United States, behind heart disease and cancer and is the leading cause of severe, long-term disability. Each year roughly 700,000 Americans experience a new or recurrent stroke. Stroke is the number one cause of inpatient Medicare reimbursement for long-term adult care. Total stroke costs now exceed $52 billion per year in US healthcare dollars. An occlusion in the cerebral vasculature can destroy millions of neurons and synapses of the brain.

### FIELD OF THE DISCLOSURES

The present disclosures relate to neurovascular medical systems of treatment, devices, methods and approaches to manufacturing devices involved in the same. More specifically, the novel device of the present invention are used to intervene and solve acute issues, as well as long term aneurysm treatments, alone or in, combination with embolic coils and other related tools of the clinician.

In terms of global populace, the largest growing demographic currently unaddressed and needing to be managed are those having strokes, or brain events based upon transient or permanent occlusion events within the relevant and proximate vasculature to that of the brain.

Since embolic coils are state of the art for brain aneurysm treatment, various approaches have tried to variegate these devices and procedures to emplace them—with limited success. The instant inventions complement existing treatments and may be used with them, seriatim or in such way as clinicians, physicians and surgeons find to be most consistent with better patient care.

### SUMMARY OF THE INVENTIONS

Briefly stated, aneurysm embolization devices made from laser cut NITINOL types of metals are highly differentiated by their form, which governs function, in addressing acute states of aneurysm to achieve improved clinical outcomes.

According to embodiments, a system as known for example from U.S. Letters Pat. Nos. 8,070,791; 8,926,680; 8,945,143; 8,574,262; 9,198,687; 9,220,522; and/or 8,088,140 to the same inventor for delivering embolic coils, and related flow diversion or other technology to the brain; is known in the art and available commercially from at least one of COVIDIEN/MEDTRONIC, STRYKER, TERUMO; BOSTON SCIENTIFIC et al., which when combined with the laser cut NITINOL device of the instant teachings creates a new standard of care.

According to embodiments there is disclosed a system for intravascular aneurysms which comprises at least a laser cut NITINOL type of device which is soft, compliant and conformable, ranging in size from at least about 1.5 mm to around 11.5 mm and deliverable through a 0.017 catheter, Or smaller.

According to embodiments, there are disclosed the systems, devices, methodologies of manufacture, deployment and quality control used in advancing the intrasacular occlusion device into patients, wherein the number of coils deployed is less than or equal to the number used in conventional procedures; and a method of making novel enhanced intrasacular occlusion devices, which comprises, in combination, providing a NITINOL tube, processing it by making it into a laser cut tube, Finishing and; Testing the same, as described or shown in this filing and all US Letters Patents referenced herein covering the devices shown in combination with prior art, and other systems for emplacement.

### BRIEF DESCRIPTION OF THE FIGURES

FIG. 1 shows a typical self-expanding NITINOL tube, according to the instant disclosure;

FIG. 2 shows a laser cut pattern, such as is typical according to the instant teachings;

FIG. 3 shows a schematized device with zones of flexure, according to this filing

FIG. 4 shows of schematics of sculpture-versions of devices according to the instant teachings;

FIG. 5 shows two views of prototypes, according to the instant inventions.

FIG. 6 shows devices of the present invention moving from a first to a second position; and

FIG. 7 shows the second or expanded configuration of subject devices cut from tubes of nitinol whereby they may be placed seriatim into the cerebral vasculature for example with coils or other therapies to treat aneurysms, AV malformations and the like.

### DETAILED DESCRIPTION OF THE INVENTIONS

The present inventor has mastered the art of delivery of medical devices to the vasculature, for example in the prior U.S. Letters Pat. Nos. 8,070,791; 8,926,680; 8,945,143; 8,574,262; 9,198,687; 9,220,522; and/or 8,088,140 to the same inventor for delivering coils and removing thrombus, several delivery systems are shown, which yield unexpectedly beneficial results with the addition of the device of the present invention.

The present inventor has discovered that another extremely helpful tool can be used in the cerebral vasculature to achieve desired clinical outcomes in a repeatable and scalable fashion and has developed systems to supply several embodiments of the invention disclosed herein provide systems, methods, and devices for the treatment of acute ischemic stroke that provide immediate blood flow restoration to a vessel occluded by a clot and, after reestablishing blood flow, address the clot itself. Immediate blood flow restoration to the neurovasculature distal to the clot can reduce the destruction of neurons and synapse of the brain that may otherwise occur if the dot is attempted to be removed without first restoring blood flow. Immediate blood flow restoration advantageously can facilitate natural lysis of the clot and also can reduce or obviate the concern for distal embolization due to fragmentation of the clot. Addressing aneurysms may be done with the present invention alone or in combination with coils and other devices.

In accordance with use with complementary devices, the clot can be addressed in-situ to reperfuse a blood vessel without occluding or blocking blood flow and without requiring the use of additional structures to address distal embolization, while an aneurysm is address to allow progressive treatments.

Using a soft and compliant intrasacular device, aneurysms can be addressed just the same way as with coils but more easily, just as prior to Applicant's aforementioned discoveries, accepted wisdom generally dictated that the thrombus should be carefully preserved so as not to disrupt or disturb the thrombus during retrieval (to avoid embolic particles from flowing distally and causing morbidity or mortality) and/or to employ distal embolic protection to capture any such embolic particles.

Several embodiments of the present invention used in complement with existing therapies and are particularly unexpected because they can be easily emplaced and stop the flow of blood and pressure. According to several embodiments used with the present invention, the release of embolic particles is, surprisingly, facilitated because blood flow (which has previously been advantageously restored) causes lysis (e.g., enzymatic digestion) of those particles such that the particles no longer pose issues distally.

Likewise, as is known embodiments of the invention provide for progressive, or modular, treatment based upon the nature of the clot. For example, the progressive treatment can comprise a three-step progressive treatment process that includes immediate restoration of blood flow, in-situ clot management, and/or clot removal depending on the particular circumstances of the treatment, The in-situ clot management can include, for example, lysis, maceration, or both. The progressive, or modular, treatment can be provided by one or more treatment devices. In some embodiments, clot removal may not be necessary due to the natural lytic destruction provided by the restoration of blood flow. In some embodiments, the progressive treatment of flow restoration, in-situ clot management, and clot removal or capture can be performed in a matter of minutes instead of hours (e.g., less than 5 minutes, less than 10 minutes, less than 15 minutes, less than 20 minutes, less than 25 minutes, less than 30 minutes, less than 45 minutes). In some embodiments, a clot management system provides treating physicians with a synergistic, two-device system optimized for both rapid reperfusion and versatile clot removal. By equipping the physician to achieve rapid perfusion, the system can help to alleviate the stress associated with racing against the clock to retrieve the clot.

In several embodiments, the outer layer of an embolus is removed via maceration and/or lysis, and the inner core of the thrombus is captured and removed. This is particularly beneficial in some embodiments because the outer layer particles are lysed by natural (or artificial) lytics or mechanical disruption and the inner core, which may be more adhesive, can be removed with minimal risk that any particles will slough off. Moreover, any small particles that are released can also be lysed by the lytic process. In some embodiments, about 30-80% of the thrombus is lysed and about 20-70% is captured and removed.

According to some embodiments of the invention, a self-expanding device, which is microcatheter-based, can be deployed across a thrombus, thereby restoring blood flow distal to the thrombus upon unsheathing. The device can then be resheathed and unsheathed one or more times to break up, or macerate, at least a portion of the clot. The device can then remain unsheathed for a period of time in order for the device to maintain restored flow, thereby facilitating natural lysis of the clot and allowing for incubation of the device within the clot to increase engagement of the clot into the surface of the device. The increased engagement can facilitate removal of the clot (if removal is necessary).

Various embodiments according to the present disclosure relate to revascularization systems and devices used to treat, among other things, ischemic stroke. Naturally, therefore, the revascularization systems and devices of several embodiments of the present disclosure are designed to be used in neuro-type applications, wherein the specifications of the present catheters and revascularization devices may be deployed in the blood vessels of the cerebral vascular system. For example, the systems and devices disclosed herein can be configured to be deployed in the cerebral arteries, including but not limited to: the anterior cerebral arteries (ACA), the anterior communicating artery, the middle cerebral arteries (MCA) (including the M1 and M2 segments), the posterior communicating arteries, the internal carotid arteries (ICA), the vertebral arteries, the basilar artery, and the posterior cerebral arteries (PCA). In some embodiments, the systems and devices are configured to be deployed in the region above the subclavian and common carotid arteries

Other embodiments of the invention are not limited to the neurovasculature and may be used in other regions, including but not limited to vessels (e.g. veins or arteries) in, to or from the heart, lungs, extremities (e.g., legs), and pelvis. Moreover, some embodiments of the invention are not limited to vascular thrombi, but instead can be directed to treatment (e.g., maceration, lysis, capture or combinations thereof) of undesired targets (e.g., gallstones, kidney stones, calcifications, cysts, fibroids, tumors, etc.). Embolic debris caused by interventions involving carotid artery stent placement and treating saphenous vein aortocoronary bypass grafts stenosis are treated according to several embodiments described herein.

In several embodiments, a method of treating a thrombus is provided. In one embodiment, the method first includes restoring blood, flow within an occluded vessel. To restore flow, a reperfusion device having a self-expanding scaffold at a distal end of a long pusher tube or wire can be temporarily inserted into the occluded vessel and advanced to the location of the thrombus. In one embodiment, the location of the thrombus refers to a Location wherein the scaffold effectively spans the thrombus (completely or substantially). Advancing the reperfusion device to the location of the thrombus can mean advancing the reperfusion device through the thrombus or to the side of the thrombus (e.g., within a microcatheter) depending on the path of least resistance and the location and morphology of the clot. In some embodiments, the reperfusion device is delivered through a microcatheter so that the self-expanding scaffold remains in a non-expanded configuration until a desired location is reached. The microcatheter can be pre-inserted or inserted together with the reperfusion device. The microcatheter can be advanced to a position wherein a distal tip of the microcatheter is located just beyond a distal end of the thrombus (e.g., within 2 cm past the thrombus, within 1 cm past the thrombus, within 5 mm past the thrombus, within 2 mm past the thrombus, aligned with the distal end of the thrombus). The reperfusion device can then be advanced within the microcatheter until the distal end of the self-expanding scaffold is aligned with, or slightly distal to, the distal end of the microcatheter.

The microcatheter can then be retracted proximally, thereby unsheathing the self-expanding scaffold and allowing the self-expanding scaffold to deploy to its expanded configuration within the thrombus. The microcatheter and the reperfusion device can be positioned such that when the self-expanding scaffold is fully deployed, it spans or substantially spans the thrombus. The self-expanding scaffold can compress the thrombus against the vessel wall, thereby creating channels within the clot for blood to flow and facilitate clot lysis. The self-expanding scaffold can comprise cells having a relatively small cell size designed to minimize, hinder, prevent, deter, or educe penetration of the thrombus, thereby maximizing the blood flow through the self-expanding scaffold. If the scaffold is not positions as effectively as desired, the microcatheter can be advanced distally to resheath the scaffold and the microcatheter and the reperfusion device can then be moved to a new position and redeployed.

In several embodiments, after a period of time after initial expansion of the self-expanding scaffold, the microcatheter can be advanced proximally to reconstrain and resheath the self-expanding scaffold and then the microcatheter can be advanced distally again to redeploy the scaffold in the same position in an effort to macerate the thrombus. The resheathing and unsheathing can be repeated one or more times. The reperfusion device can then be removed by advancing the microcatheter distally to resheath the scaffold and then withdrawing the reperfusion device from the body (with or without the microcatheter).

It is respectfully submitted that the following constitutes invention, because it has addressed series of problems that have yet to be adequately addressed among the prior art.

Looking at the appendices, one notices that although the sets of approaches illustrated demonstrate that there is a longstanding need for solutions to the instant problems, none are obvious or forthcoming based upon these other systems.

For example, each of the shown devices needs multiple and often redundant types of passes to obstruct the blood flow within the sac. The “flow diverter” approach (and most others) requires the use of anti-coagulants for life.

Likewise, the WEB brand of device and LUNA (NFocus/Covidien) device are not easily position, tracked or emplaced optimally.

Similarly, clinicians and though leaders have challenges getting the MEDINA MEDICAL device to be properly positioned, or to block flow enough to have not to repeat and repeat and repeat to achieve the desired clinical endpoints.

Each of these issues is managed according to the instant teachings.

The present inventor has discovered how to address brain aneurysms with e flexible and compliant approach.

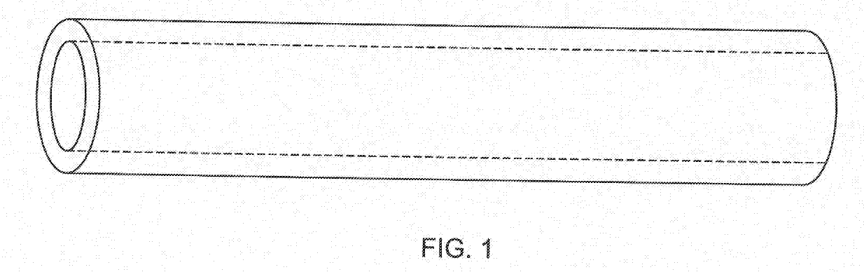

Turning to FIG. 1-7, those skilled in the art understand that the schematic for a laser cut NITINOL tube is used to show a cylindrical flow path there through, the width, flexural modulus and degree of softness are generally well know and/or explained in U.S. Letters Pat. No. 8,070,791; 8,926,680; 8,945,143; 8,574,262; 9,198,687; 9,220,522; and/or 8,088,140 to the same inventor.

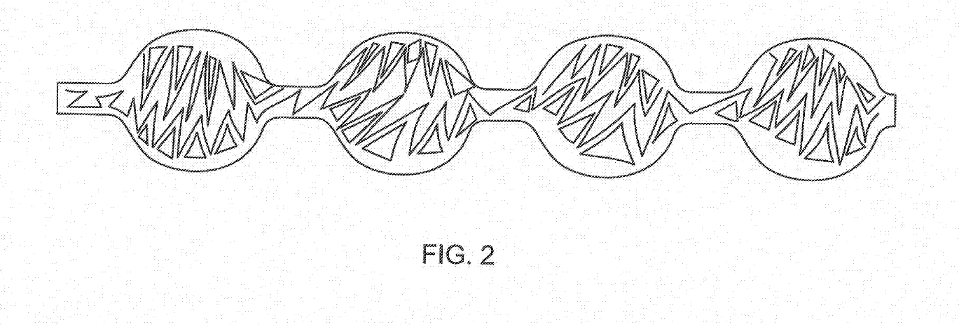

FIG. 2 shows a typical laser cut pattern, such as is created according to the instant teachings, namely, the present devices are made to be soft and compliant with the walls of the vessels in which they are emplaced. To do this chronic outward radial force is managed with various cell structures to allow for zones of flexure, whereby the devices are bent and folded prior to deployment, as known to those skilled in the art of NITINOL.

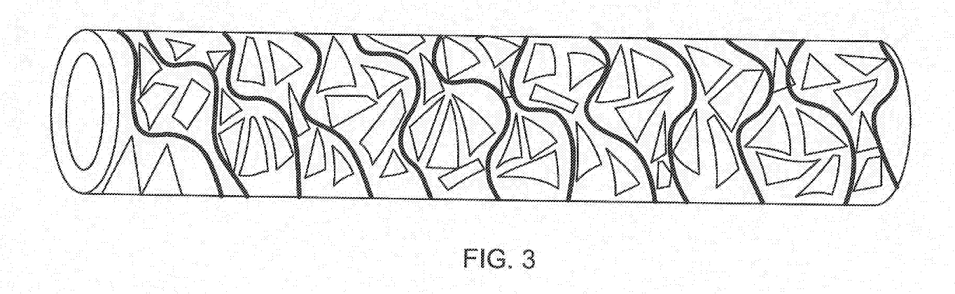

FIG. 3 shows a schematized device with zones of flexure, according to this filing, with the darkened hands indicating edges and borders of respective zones, allowing for a flower-petal like arrangement and folding of the device for delivery.

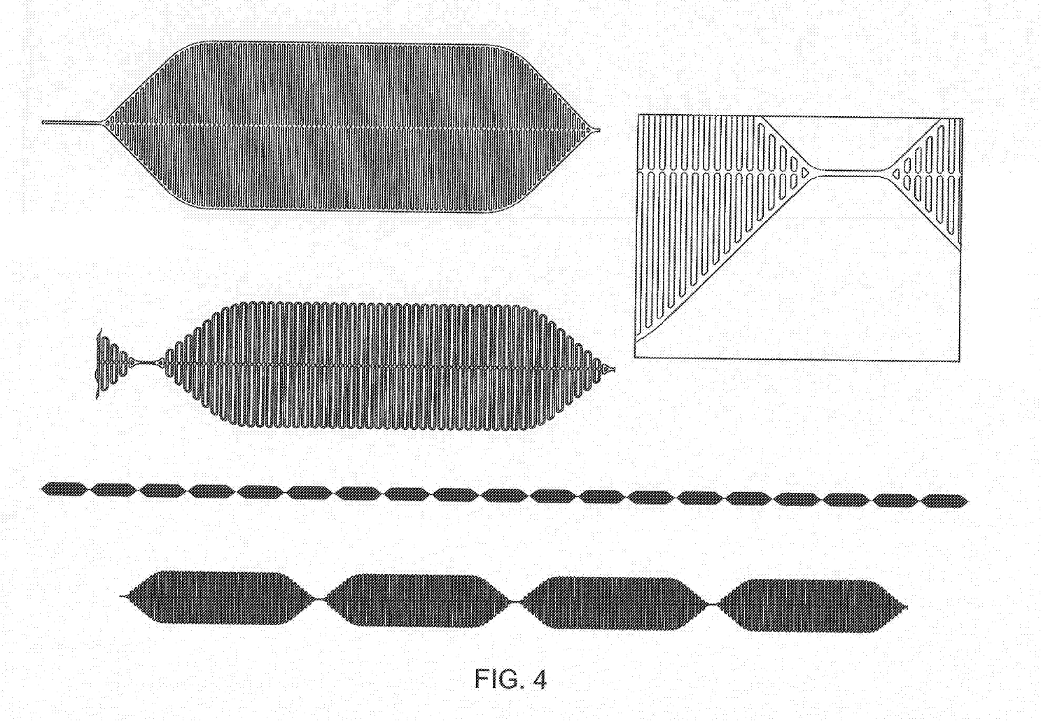

FIG. 4 likewise shows a series of schematics of sculpture-versions of devices according to the instant teachings, details shown of junctures and flew points, whereby the same can be loaded and mounted for delivery.

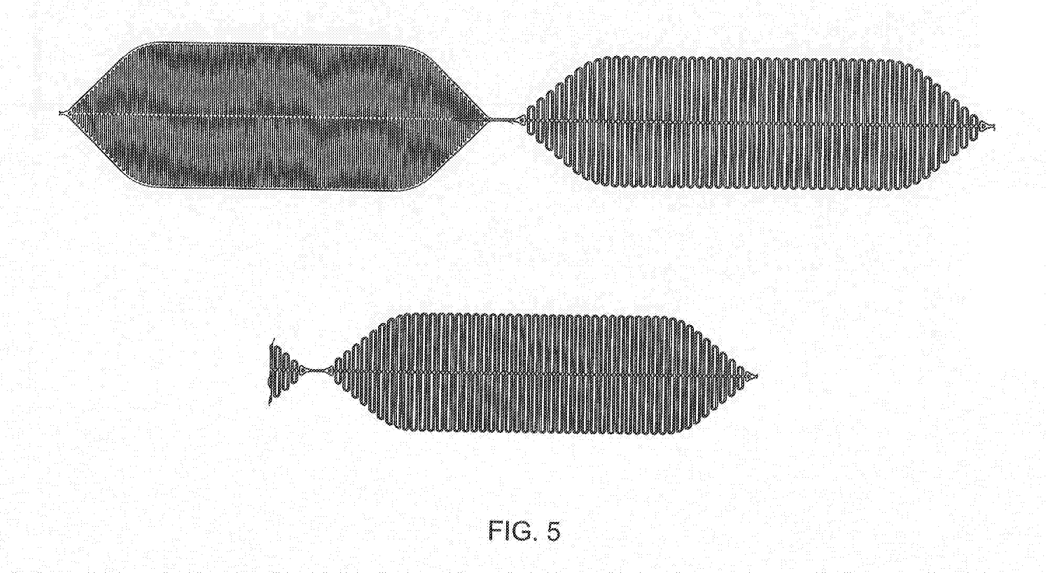

FIG. 5 shows two views of prototypes, according to the instant inventions, showing typical cells structures which allow for rapid deployment and correct placement.

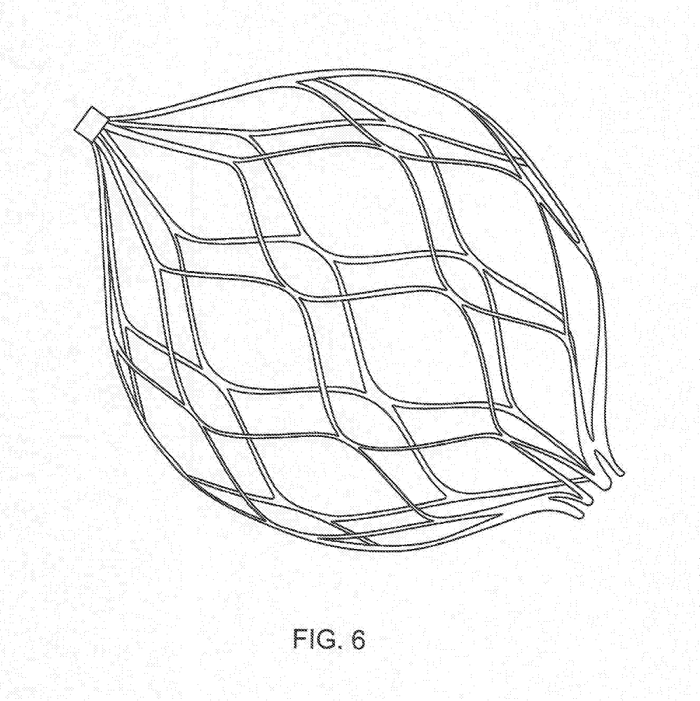

FIG. 6 shows devices of the present invention moving from a first to a second position; and

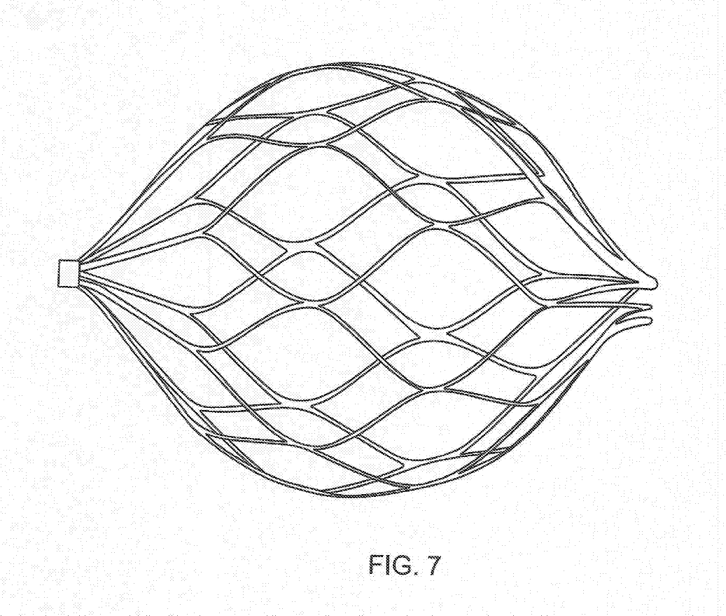

FIG. 7 shows the second expanded configuration of subject devices cut from tubes of nitinol whereby they may be placed seriatim into the cerebral vasculature for example with coils or other therapies to treat aneurysms, AV malformations and the like.

Likewise, referring back to FIG. 1 through FIG. 3, steps in the process for making the instant device are shown. Those skilled in the art understand both how to procure, cut and shape NITINOL tubes, however, no teachings have shown how to deploy them as shown in avoid prior art pitfalls. FIG. 4 and FIGS. 5-7 each show laser-cut nitinol tubes, which are folded into aneurysm sacs to occlude the same. Flattened points of joinder permit foldings of many devices seriatim into an aneurysm.

Likewise those skilled understand that no anti-coagulant is needed with the instant systems and that amorphous and hard to manage necks can be treated.

1. A novel INTRASACULAR OCCLUSION DEVICE, comprising a system for addressing intravascular aneurysms which further comprises, in combination:
at least a laser cut NITINOL type of device,
ranging in size from at least about 1.5 mm to around 11.5 mm;
and deliverable through a 0.017 catheter and other microcatheter assemblies having smaller profiles.

2. The device of claim 1, which further comprises, in combination:
at least a laser cut NITINOL type of device,
ranging in size from at least about 2 mm to around 10 mm;
and deliverable through a 0.017 catheter and devices having smaller profiles.

3. The devices of claim 2, being more soft, compliant and conformable than conventional laser cut articles used for minimally invasive surgical interventional procedures.

4. The devices of claim 3, wherein the resultory system improves aneurysm neck support and coverage over conventional devices in situ.

5. The devices of claim 4, used in complement with embolic coils, wherein the number of coils deployed is less than or equal to the number used in conventional procedures.

6. The devices of claim 5, wherein said devices are compatible with platinum coils and do no require anti-coagulant therapy.

7. The devices of claim 6, which are radiopaque and can be visualized during and post-procedures.

8. The devices of claim 7, which can be manufactured at scale.

9. A method of making a making novel intrasacular occlusion device, which comprises, in combination:
providing at least a NITINOL tube;
processing it by making it into a laser cut tube;
finishing the said laser cut tube and;
testing the same, to ensure compliance with quality control needed for neurovascular emplacement; and repeating any of the steps.

10. The method of claim 9 whereby the laser cut NITINOL tube has a pattern that allows it to be more flexible, soft and compliant than expected when emplaced.

11. The method of claim 10, whereby zones of flexure permit the laser cut NITINOL tube to be expanded from a first to a second position with the brain, for emplacement within the sac of an aneurysm without any insult or injury to the surrounding vasculature.

12. The method of claim 11, whereby unexpectedly difficult placements are achieved within the brain of a patient, whereby fewer complementary devices, such as coils are used.

13. The method of claim 9, whereby the laser cut NITINOL tube is able to be emplaced through a delivery catheter having a profile less than or equal to 0.017.

14. The devices of claim 8, whereby the devices move from a first, retracted, to a second, expanded configuration.

15. The devices of claim 14, emanating from a flattened section during the first configuration.

16. The devices of claim 15, expandable and compressable in a second configuration on to be used to pack an aneurysm.

17. The devices of claim 16, adequately compliant to be used in multiple versions, along or with other therapies, in situ.

18. The devices of claim 17, effective to counter the growth of aneurysms in the brain.
